(19) United States
(12) Patent Application Publication
(10) Pub. No.: US 2010/0034763 A1
(43) Pub. Date: Feb. 11, 2010

(54) Skin Lightening Composition Comprising CO2 Extracts

(75) Inventor: Stephen Alan Madison, Newtown, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/185,885

(22) Filed: Aug. 5, 2008

(51) Int. Cl. A61K 8/34; A61K 8/97; A61Q 19/02

(52) U.S. Cl. 424/62

(57) ABSTRACT

Skin lightening additives and skin lightening compositions with carbon dioxide obtained plant extracts are described. The compositions are suitable for topical application and may comprise CO₂ obtained Chamomile extract.

### FIELD OF THE INVENTION

This invention is directed to a composition for lightening skin. More particularly, the present invention is directed to a cosmetic composition comprising a carbon dioxide obtained plant extract. The carbon dioxide obtained plant extract comprises a terpene and is substantially free of allergens like anthecotulide. The carbon dioxide obtained plant extract is natural, lipophilic and easy to formulate in compositions desired for topical application wherein such compositions unexpectedly result in excellent skin lightening benefits after use.

### BACKGROUND OF THE INVENTION

Many people are concerned with the degree of pigmentation of their skin. For example, people with age spots or freckles often wish for such pigmented spots to be less pronounced. Others wish to reduce the skin darkening caused by exposure to sunlight or to lighten their natural skin color. To meet this need, many attempts have been made to develop products that reduce pigment production in melanocytes. However, the substances identified thus far tend to have either low efficacy or undesirable side effects, such as, for example, toxicity or skin irritation.

There is an increasing interest to develop skin lightening compositions that yield excellent lightening results yet do not yield undesirable side effects. This invention, therefore, is directed to skin lightening compositions that comprises carbon dioxide obtained plant extract. The skin lightening composition of the present invention comprise a carbon dioxide obtained plant extract comprising a terpene and the composition is substantially free of allergens like antheculide. Such a composition unexpectedly results in excellent skin lightening benefits after use.

### ADDITIONAL INFORMATION

Efforts have been disclosed for making skin care cosmetic compositions. In U.S. Pat. No. 6,875,425, skin lightening agents with 4-substituted resorcinol derivative compounds are described.

Other efforts have been disclosed for making skin treatment compositions. In U.S. Pat. Nos. 7,250,158 and 7,247,294, methods for treating skin with lightening agents are described.

Still other efforts have been disclosed for treating skin. In U.S. Pat. No. 5,998,423, compositions with polycyclic nitrogen heterocycles are described.

Even other efforts have been disclosed for lightening skin. In U.S. Published Patent Application Nos. 2006/0110341 A1 and 2006/0153787 A1, compositions with skin lightening agents are described.

None of the additional information above describes a skin lightening composition that comprises, as a lightening additive, a carbon dioxide obtained plant extract wherein the extract comprises a terpene and is substantially free of allergens.

### SUMMARY OF THE INVENTION

In a first aspects the present invention is directed to a skin lightening additive comprising a carbon dioxide obtained plant extract wherein the extract comprises a terpene.

In a second aspect, the present invention is directed to a skin lightening composition comprising the skin lightening additive of the first aspect of this invention.

In a third aspect, the present invention is directed to a method for lightening skin with the composition of the second aspect of this invention.

All other aspects of the present invention will more readily become apparent upon considering the detailed description and examples which follow.

As used herein, a decrease in melanin content means a melanin content decrease when comparing two (2), three week old MatTek MelanoDerm™ cultures that have not been treated with a composition comprising the skin lightening additive of this invention to two (2), three week old MatTek MelanoDerm cultures that have been treated with a composition comprising the skin lightening additive of this invention wherein treated means:
(a) placing the MelanoDerm culture within a six (6) well tissue culture dish and set about 0.3 cm off of the tissue culture dish;
(b) subjecting the MelanoDerm culture to 0.004-0.4% by weight compositions having the skin lightening additive of this invention, the composition being one prepared from a solution of skin lightening additive and carrier (erg., dimethyl sulfoxide, to a final concentration of 0.1% DMSO) having been diluted with Dulbecco's Modified Eagle Media; and
(c) comparing the treated and untreated cultures by obtaining average melanin content (expressed in micrograms) by extracting melanin from MelanoDerm and obtaining absorbance readings at 490 nm (OD490) using a commercially available spectrophotometer like a Hach Spectrophotometer.

Skin lightening composition, as used herein, is meant to include a composition for topical application to skin of mammals, especially humans. Such a composition may be generally classified as leave-on or rinse off, and is meant to include conditioners or tonics, lipsticks, color cosmetics, and general topical compositions that in some fashion and at the very least, reduce the effect of melanin on skin color. Lightening, as used herein, is meant to mean the lightening or whitening of skin directly as well as the lightening or whitening of spots on the skin, like age spots and freckles. Dulbecco's Modified Eagle Media means the nutrient solution sold by MatTek Corporation and treated and used according to instructions supplied with the product commercially identified as MEL30010BBLLMM. Skin lightening additive means a component suitable to result in physical, but especially biological whitening (i.e., a reduction in melanin production) whereby the skin lightening composition of this invention unexpectedly results in a decrease in melanin content which is at least about 20%, and preferably, from about 25 to about 70%, and most preferably, from about 25 to about 65% lower when comparing MelanoDerm cultures treated with the same to MelanoDerm cultures that have not been subjected to the composition of the present invention. MelanoDerm means the product having normal, human-derived epidermal keratinocytes and melanocytes which have been cultured to form a multilayered, highly differentiated model of the human epidermis, all of which is made commercially available by MatTek Corporation. Comprising, as used herein, is meant to include consisting essentially of, and consisting of, and carbon dioxide obtained is meant to mean extracted from its origin with carbon dioxide where such an extraction can include processes that use supercritical carbon dioxide. Substantially free of means less than about 0.15 percent by weight, and preferably, from 0 to less than about 0.1% by weight. Terpene, as used herein, is meant to mean comprising an isoprene unit or a derivative thereof. Plant extract, as used herein, means the extract obtained from leaf, stem, root and/or flower of the plant.

The skin lightening composition of the present invention can be in the form of a liquid, lotion, cream, serum, gel, soap bar or toner, or applied via a face mask or patch. Skin is meant to include skin on the face, neck, chest, back, arms, hands, legs and scalp. All ranges identified herein are meant to implicitly include all ranges subsumed therein, if, for example, reference to the same is not explicitly made.

### DETAILED DESCRIPTION OF THE PREFERRED EMBODIMENTS

The only limitations with respect to the skin lightening additives that may be used in this invention are that the same may be used in topical applications and that they are obtained by an extraction process that uses carbon dioxide to obtain a plant extract. Such carbon dioxide obtained plant extracts preferably comprise a terpene and May be recovered from plants like those generally classified in the family Asteraceae. Preferred plants for obtaining the extracts that may be used in this invention include those in the Matricaria, Anthemis, Atemisia and Achillea genuses. In an especially preferred embodiment, the plants that are suitable for obtaining the extracts comprising a terpene are Chamomile, Yarrow and wormwood plants. When preparing the carbon dioxide obtained plant extract of this invention, plant material may be placed in a pressure vessel and carbon dioxide may be passed through in order to obtain the desired skin lightening additives. Another alternative is to steep the plant material in liquid carbon dioxide. The extracts, when obtained by carbon dioxide extraction, are neat (i.e., not carried in an extraction oil), substantially free of allergens, lipophilic and convenient for formulating with. A more detailed description of plant extraction with carbon dioxide may be found in U.S. Pat. No. 4,554,170, the disclosure of which is incorporated herein by reference. Moreover, plant extracts obtained by CO₂ extraction can be purchased from suppliers like Flavex® Naturextrakte GmbH.

In a most preferred embodiment, the terpene present in the extract employed in this invention is bisabolol, farnesene, chamazulene, ocimene, matricine, bisabolol oxide, spathulenol, mixtures thereof or the like. When employed, the amount of farnesene in the extract is typically from about 0.5 to about 15, and preferably, from about 0.75 to about 10, and most preferably, from about 2 times greater to about equal the amount of bisabolol in the extract. Often, however, the amount of bisabolol oxide in the extract is from about 0.001 to about 5 times greater, and preferably, from about 0.01 to about 3.5, and most preferably, from about 0.2 to about 1.5 times greater than the amount of bisabolol in the extract.

In general, the amount of terpene present in the skin lightening composition of the present invention is from about 0.005 to about 2%, and preferably, from about 0.05 to about 1.5%, and most preferably, from about 0.1 to about 1.2% by weight, based on total weight of the skin lightening composition and including all ranges subsumed therein.

Typically, the skin lightening composition of the present invention comprises from about 0.01 to about 15%, and preferably, from about 0.02 to about 10%, and most preferably, from about 0.05 to about 5% by weight carbon dioxide obtained plant extract, based on total weight of the skin lightening composition and including all ranges subsumed therein.

It should be known that commercially acceptable and conventional vehicles may be used, acting as diluents, dispersants and/or carriers for the skin lightening additives described herein and for any other optional but often preferred additives. Therefore, the cosmetically acceptable vehicle suitable for use in this invention may be aqueous-based, anhydrous or an emulsion whereby a water-in-oil or oil-in-water emulsion is generally preferred. If the use of water is desired, water typically makes up the balance of the cosmetic composition, and preferably, makes up from about 5 to about 99%, and most preferably, from about 40 to about 80% by weight of the skin lightening composition, including all ranges subsumed therein.

In addition to water, organic solvents may be optionally included to act as carriers or to assist carriers within the compositions of the present invention. Illustrative and non-limiting examples of the types of organic solvents suitable for use in the present invention include alkanols like ethyl and isopropyl alcohol, mixtures thereof or the like.

Other optional additives suitable for use include ester oils like isopropyl myristate, cetyl myristate, 2-octyldodecyl myristate, avocado oil, almond oil, olive oil, neopentylglycol dicaprate, mixtures thereof or the like. Typically, such ester oils assist in emulsifying the skin lightening composition of this invention, and an effective amount is often used to yield a stable, and most preferably, water-in-oil emulsion.

Emollients may also be used, if desired, as carriers within the skin lightening composition of the present invention. Alcohols like 1-hexadecanol (i.e., cetyl alcohol) are often desired as are the emollients generally classified as silicone oils and synthetic esters. Silicone oils suitable for use include cyclic or linear polydimethylsiloxanes containing from 3 to 9, preferably from 4 to 5, silicon atoms. Nonvolatile silicone oils useful as an emollient material in the skin lightening composition described herein include polyalkyl siloxanes, polyalkylaryl siloxanes and polyether siloxane copolymers. The essentially non-volatile polyalkyl siloxanes useful herein include, for example, polydimethylsiloxanes.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterol esters, of which cholesterol fatty acid esters are examples.

Emollients, when used, typically make up from about 0.1 to about 50% by weight of the skin lightening composition, including all ranges subsumed therein.

Fatty acids having from 10 to 30 carbon atoms may also be included as cosmetically acceptable carriers within the composition of the present invention. Illustrative examples of such fatty acids include pelargonic, lauric, myristic, palmitic, stearic, isostearic, oleic, linoleic, arachidic, behenic or erucic acid, and mixtures thereof. Compounds that are believed to enhance skin penetration, like dimethyl sulfoxide, may also be used as an optional carrier.

Humectants of the polyhydric alcohol type may also be employed in the skin lightening composition of this invention. The humectant often aids in increasing the effectiveness of the emollient and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycerol or sodium hyaluronate. The amount of humectant may range anywhere from 0.2 to 25%, and preferably, from about 0.5 to about 15% by weight of the skin lightening composition, based on total weight of the composition and including all ranges subsumed therein.

Thickeners may also be utilized as part of the cosmetically acceptable carrier in the skin lightening composition of the present invention. Typical thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

Surfactants may also be present in skin lightening composition of the present invention. Total concentration of the surfactant will range from about 0 to about 40%, and preferably, from about 0 to about 20%, optimally from about 0 to about 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

Perfumes may be used in the composition of this invention. Illustrative non-limiting examples of the types of perfumes that may be used include those described in Bauer, K., et al., Common Fragrance and Flavor Materials, VCH Publishers (1990).

Illustrative yet non-limiting examples of the types of fragrances that may be used in this invention include myrcene, dihydromyrenol, citral, tagetone, cis-geranic acid or citronellic acid, mixtures thereof or the like.

Preferably, the amount of fragrance employed in the skin lightening composition of this invention is in the range from about 0.0% to about 10%, more preferably, about 0.00001% to about 5 wt %, most preferably, about 0.0001% to about 2%

Various types of optional additional active ingredients may be used in the skin lightening compositions of the present invention Actives are defined as skin benefit agents other than emollients and other than ingredients that merely improve the physical characteristics of the composition. Although not limited to this category, general examples include talcs and silicas, as well as alpha-hydroxy acids, beta-hydroxy acids, peroxides, zinc salts, and sunscreens.

Beta-hydroxy acids include salicylic acid, for example. Zinc pyrithione is an example of the zinc salts useful in the skin lightening composition of the present invention.

Sunscreens include those materials commonly employed to block ultraviolet light. Illustrative compounds are the derivatives of PABA, cinnamate and salicylate. For example, avobenzophenone (Parsol 1789®), octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation. Additives that reflect or scatter the suns rays may also be employed. These additives include oxides like zinc oxide and titanium dioxide.

Many topical compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Still other optional ingredients that may be used with the skin lightening composition of this invention include dioic acids (e.g., malornic acid, sebacic acid), antioxidants like vitamin E, other vitamins, like vitamin C and its derivatives, recorcinols and its derivatives (including those esterified with, for example, ferulic acid, vanillic acid or the like) and retinoids, including retinoic acid, retinal, retinol and retinyl esters, conjugated linoleic acid, petroselinic acid and mixtures thereof, as well as any other conventional ingredients well known for wrinkle-reducing, skin whitening (especially, niacinamide and/or cis-en dicycloether), anti-acne effects and reducing the impact of sebum.

The skin lightening composition of the present invention is intended for use primarily as a product for topical application to human skin, especially and at least as a product for lightening the skin. Thus, the inventor has discovered that the described extracts have excellent skin lightening capabilities whereby the same may be employed as skin lightening additives in compositions that are applied topically to areas of the skin where lightening or whitening is desired. Other benefits may include skin moisturizing, decreasing the effect of sebum on the skin and skin wrinkle reducing. In an especially preferred embodiment, the composition of the present invention has a pH from about 4.5 to about 7.5, including all ranges subsumed therein.

When making the skin lightening composition of the present invention, the desired ingredients are mixed, in no particular order, and usually at temperatures from about ambient to about 80° C. and under atmospheric pressure.

The packaging for the composition of this invention can be a patch, bottle, tube, roll-ball applicator, propellant driven aerosol device, squeeze container or lidded jar.

The examples which follow are provided to illustrate and facilitate an understanding of the invention. The examples are not intended to limit the scope of the claims.

### EXAMPLE

Commercially available human skin equivalents (MelanoDerm™ from MatTek) were obtained for testing the impact of carbon dioxide obtained plant extract on melanogenesis. Solutions having a final concentration of 0.004-0.4% by weight were prepared in dimethyl sulfoxide stock solution (final concentration 0.1% by weight) and dosed ten (10) times in a three (3) week period into the media of the MelanoDerm cultures. The media consisted of commercially available basal Dulbecco's Modified Eagles media, prepared and treated in the manner set forth in the manufacturer's instructions. For long term maintenance of the MelanoDerms, the basal media was supplemented with bFGF and alpha MSH to stimulate melanocyte growth and melanogenesis. Each treatment condition was done in duplicate and three (3) sets were made for each treatment, as well as for a control (culture not treated with the carbon dioxide obtained plant extract). The cultures were maintained at a temperature of about 37° C. and incubated in a humidified, 5% CO₂ incubator during the dosing period, but removed while being dosed.

After a three (3) week period, the MelanoDerm cultures were removed and solubilized in a centrifuge tube containing 250 microliters of Solvable reagent (GNE9100, Packard) for sixteen (16) hours (overnight) in a 60° C. oven. Following solubilization, the centrifuge tube containing the sample was spun at 12,000 g for five (5) minutes. Two hundred (200) microliters of supernatant were removed and placed in a ninety-six (96) well plate. A spectrophotometer was used to measure the absorbance of the supernatant at 490 nm. A standard curve using synthetic melanin was set up in parallel to allow quantitation of melanin, in micrograms, of the samples. The results are provided below:

TABLE

| | | | Standard |
|---|---|---|---|
| Active | Average MC* | ΔMC | Deviation |
| Control | 68.8 | — | |
| Carbon dioxide** | | | |
| obtained | | | |
| Chamomile Extract: | | | |
| i) | 49.6 | 19.2 | 4.7 |
| ii) | 41.2 | 27.6 | 3.7 |
| iii) | 46.5 | 22.3 | 4.6 |
| Control | 77.2 | | |
| Greentech USA, Inc. EG 004 | | | |
| Phytelene Chamomile | | | |
| (glycol extracted) | | | |
| ii) | 60.8 | 16.4 | 4.9 |
| iii) | 78.2 | −1.0 | 3.5 |
| Symrise A.G. Extrapone | | | |
| Chamomile BR (water/ | | | |
| glycol extracted) | | | |
| ii) | 64.9 | 12.3 | 16.0 |
| iii) | 66.8 | 10.4 | 2.6 |

*MC = melanin content
**= Chamomile (CO₂ obtained, commercially available by Flavex ®) extract where farnesene, bisabolol, and bisabolol oxide are in the weight ratio of about 1 to 1 to 0.5.
i) = 0.004% by weight extract
ii) = 0.04% by weight extract
iii) = 0.4% by weight extract

The results, as they relate to MelanoDerm cultures, show that skin lightening compositions with the carbon dioxide obtained plant extract of this invention unexpectedly result in excellent skin lightening results.

What is claimed is:

1. A skin lightening additive comprising a carbon dioxide obtained plant extract wherein the extract comprises a terpene.

2. The skin lightening additive according to claim 1 wherein the terpene comprises bisabolol, farnesene, azulene, ocimene, matricine, bisabolol oxide, spathulenol, or mixtures thereof.

3. The skin lightening additive according to claim 1 wherein the terpene comprises farnesene and matricine.

4. The skin lightening additive according to claim 3 wherein farnesene is present at an amount that is from about 5 to about 25 times greater than an amount of matricine.

5. The skin lightening additive according to claim 1 wherein the additive further comprises cis-en dicycloether.

6. The skin lightening additive according to claim 1 wherein the additive further comprises niacinamide.

7. A skin lightening composition comprising:
a) a skin lightening additive comprising a carbon dioxide obtained plant extract comprising a terpene; and
b) carrier.

8. The skin lightening composition according to claim 7 wherein the extract is from a plant classified in family Asteraceae.

9. The skin lightening composition according to claim 7 wherein the extract is from a plant classified in genus Matricaria, Anthemis, Attemisia, Achillea or a mixture thereof.

10. The skin lightening composition according to claim 7 wherein the extract is from Chamomile, Yarrow, wormwood plant or a mixture thereof.

11. The skin lightening composition according to claim 7 wherein the skin lightening additive further comprises niacinamide.

12. The skin lightening composition according to claim 7 wherein the terpene comprises farnesene and matricine.

13. The skin lightening composition according to claim 12 wherein farnesene is present at an amount that is from about 5 to about 25 times greater than an amount of matricine.

14. The skin lightening composition according to claim 7 wherein the composition further comprises cis-en dicycloether.

15. A method for lightening skin comprising the step of contacting the skin with the skin lightening composition of claim 7.
